(19) United States
(12) Patent Application Publication
(10) Pub. No.: US 2006/0280703 A1
(43) Pub. Date: Dec. 14, 2006

(54) Antimycotic nail varnish

(76) Inventors: Pascal LeFrancois, Lavaur (FR); Roger Navarro, Pamiers (FR); Marlene Delaunois, Cessales (FR)

Correspondence Address: BIRCH STEWART KOLASCH & BIRCH, PO BOX 747, FALLS CHURCH, VA, 22040-0747, US

(21) Appl. No.: 10/565,936

(22) PCT Filed: Jul. 28, 2004
(86) PCT No.: PCT/FR04/02019

§ 371 Date: Jun. 16, 2006

(30) Foreign Application Priority Data
Jul. 29, 2003 (FR) 0309303

(51) Int. Cl. A61K 8/49; A61K 31/44

(52) U.S. Cl. 424/61; 514/345

(57) ABSTRACT

Antimycotic nail varnish containing 2 to 80 wt. %, in relation to the amount of volatile compounds, of at least one 1-hydroxy-2-pyridone of general formula (I) as an antimycosic active substance and 0.1 to 20 wt %, in relation to the amount of volatile compounds, of an absorption promoter.

A subject matter of the present invention is a nail varnish exhibiting an antimycotic activity.

Conditions of the nails due to fungi (onychomycoses) are stubborn forms of diseases which it has not been possible to date to satisfactorily treat.

The name onychomycoses embraces various types of mycoses of the nails, including those brought about by dermatophytes, which are the most difficult to treat, while mycoses of the nails brought about by yeasts have to date been able to be more easily treated.

Various routes have been used for the treatment of these mycoses, but without real success.

The systemic method, which consists of the oral administration of antifungal agents, is a long-term treatment which can result in side effects and in the selection of resistant strains. These treatments, which require the administration of high doses of therapeutic agent, are consequently preferably reserved for severe cases of onychomycoses. The antimycotic molecules used for the oral route are itraconazole, terbinafine and fluconazole (Effendy I., Therapeutic Strategies in Onychomycosis. Journal of the European Academy of Dermatology and Venereology, 1995, S3-S10).

The removal of the nails by surgery or by the action of chemicals is a very aggressive method which gives no guarantee that the nails will regrow in their normal form; on the contrary, the nails which regrow are frequently deformed.

The local treatment of the nails with specific substances possessing an antimycotic action has also been envisaged.

Thus, in a combined treatment, the nails have been treated first with solutions of substances possessing an antimycotic action and then dressings with creams have been applied overnight. This method of treatment is very unpleasant and psychologically testing for the patients. This is because, first, the treatment of the nails with solutions is necessary several times daily and, secondly, the nails have to be provided with dressings throughout the night. In addition, it is necessary to constantly file the diseased nails, which not only is tiresome but also contributes to the spread of the pathogenic microorganisms. Finally, this treatment, which lasts several months, is often not pursued by the patients who, on the contrary, lose heart and become careless, which results in the failure of the treatment.

The success of this treatment is additionally compromised in that the solutions and the creams are usually miscible with water or hydrophilic and can consequently be removed from the surface of the nail or be carried away by dissolution out of the nail when washing and thus have to be reapplied.

More recently, methods involving a nail varnish comprising a substance possessing antimycotic activity have been developed.

The latter methods are the most effective. The recent development of novel antimycotic agents in the form of nail varnishes has made it possible to obtain very good results in the case of the treatments of moderate infections without recourse to surgery (Goettman S., Treatment of Dermatophytic Onychomycosis. Journal de Mycology Médicale, 1995, 5 (1) 11-16). These varnishes have the advantage of being easy to apply and of forming a water-insoluble film at the surface of the nail which makes possible the release of the antimycotic agent within the nail. The antimycotic molecules formulated in these nail varnish bases are amorolfine, ciclopirox olamine and the bifonazole/urea combination (De Chauvin M. F., Baran R. and Charasse D. Onychomycosis. III: Treatment. Journal de, Mycologie Medicale, 2001, 11 (4), 205-215; Fleckman P., Onychomycosis: Diagnosis and Topical Therapy. Dermatologic Therapy, 2002, 15 (2), 71-77).

Patent EP 855 397 discloses antimycotic agents which comprise azole derivatives, in particular imidazole and triazole derivatives, as active substances. These antimycotic agents can be applied in the form of a thin water-soluble film, can display a deposit effect and can make possible a short-term treatment. They are appropriate for the treatment of mycoses of the nails and can be used both in solutions and in aerosols which form, after drying, a thin water-soluble film. The use of such binders in water obviously has the effect that the agent applied is more or less removed every time washing is carried out.

In order to facilitate the penetration of the antimycotic agents through the nail, these varnishes often comprise keratolytic substances, such as urea.

Application EP 226 984 discloses a nail varnish comprising a water-insoluble film-forming agent and an antimycotic agent. The varnish can comprise urea but the amount of urea is not specified.

Application EP 1 138 314 discloses a prolonged-release film-forming composition for nail varnishes comprising an antifungal agent, urea, a humectant, such as glycerol or sorbitol, water, a film-forming agent and a solvent comprising at least one volatile component. The joint use of urea and of glycerol improves the trans-nail passage of the antifungal molecule and the presence of glycerol makes it possible to use the urea at concentrations of less than 1% with respect to the total weight of the composition.

German application DE 10061801 discloses a pharmaceutical composition which comprises a hydrophilic film-forming agent, water and urea. The hydrophilic film-forming agent is polyvinylpyrrolidone in concentrations of 15-25% by weight. The urea is present at a very high concentration, more particularly 75-85% by weight.

Patent U.S. Pat. No. 5,346,692 discloses a nail varnish formulation for treating onychomycoses which comprises a film-forming agent, an antimycotic agent, urea and a very specific mixture of solvents; the formulations comprise a high proportion of urea, preferably 15 to 60% with respect to the nonvolatile compounds.

In view of the state of the art described above, the present invention provides a nail varnish for treating onychomycosis which is capable of being applied topically to the infected region of the nail in the form of a film, which makes possible good release of the active principle and good diffusion of this active principle over the infected regions without attacking the healthy regions of the nail and which comprises a low amount of an absorption-facilitating agent, preferably urea, despite the absence of a humectant.

The subject matter of the invention is thus a nail varnish possessing an antimycotic action comprising:
a) 2 to 80% by weight, with respect to the amount of volatile compounds, of at least one 1-hydroxy-2-pyridone of general formula (I) 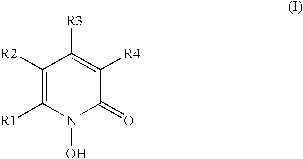
   in which
R1 represents either a straight or branched alkyl group having from 6 to 9 carbon atoms or a cycloalkyl group having from 3 to 6 carbon atoms,
one of the substituents R2 and R4 represents a hydrogen atom and the other represents a hydrogen atom or a group chosen from the methyl and ethyl groups, and
R3 represents a straight or branched alkyl group having 1 or 2 carbon atoms,
in the free form or in the form of a physiologically acceptable salt, as substance possessing an antimycotic action,
b) from 0.1 to 20% by weight, with respect to the amount of volatile compounds, of a promoter of absorption of the active substance chosen from the group comprising urea, ammonium sulfite, esters and salts of thioglycolic acid, allantoin, enzymes and salicylic acid,
c) 5 to 40% by weight, with respect to the amount of volatile compounds, of a film-forming agent which is soluble in polar solvents, and optionally
d) a physiologically acceptable solvent, as well as, optionally,
e) additives acceptable in cosmetology.

In an advantageous embodiment of the invention, the nail varnish is characterized in that the active substance possessing an antimycotic action corresponds to the formula (I) in which R2 and R4 are each a hydrogen atom.

In a particularly advantageous embodiment of the invention, the nail varnish comprises an active substance possessing an antimycotic action corresponding to the formula (I) in which R1 represents a cycloalkyl group having from 3 to 6 carbon atoms, preferably a cyclohexyl group or a —CH₂—CH(CH₃)—CH₂—C(CH₃)₃ group.

In another particularly advantageous embodiment of the invention, the absorption promoter is urea.

In the nail varnish according to the invention, with respect to the amount of the nonvolatile components, that is to say to the sum of the film-forming agents, of the pigments optionally present, of the plasticizers and of the other nonvolatile additives, and of the active substance, the latter is generally present in an amount ranging from 2 to 80% by weight, preferably from 10 to 60% by weight and in particular from 30 to 40% by weight.

In the nail varnish according to the invention, the active substance is present in an amount ranging from 0.5 to 20% by weight, preferably from 2 to 15% by weight, with respect to the amount of nonvolatile components.

The nail varnishes according to the invention additionally comprise, as essential components, one or more film-forming agents which form a thin water-insoluble film on the nail after the preparation has dried.

These film-forming agents are chosen from the group consisting of acrylic/methacrylic copolymers, polymethacrylate/butyl acrylates, acrylic/acrylate copolymers, polyvinyl alcohol, polyvinylpyrrolidones, polyvinylpyrrolidone/vinyl acetates and vinyl-pyrrolidone/dimethylaminoethyl methacrylates.

It is known that the combination of the solvents is of dominating importance with regard to the drying time, the ease of application with a brush and other important properties of the varnish or of the thin varnish film.

According to the present invention, the system of solvents is preferably composed of an optimum mixture of solvents possessing a low boiling point (less than 100° C.) and of solvents possessing a moderate boiling point (of between 100 and up to 150° C.); optionally with a small proportion of solvents possessing a high boiling point (of between 150 and up to 200° C.).

The choice will preferably be made, as solvent, of the water/ethanol, water/isopropyl alcohol or water/butanol mixtures in ratios ranging from 10/90 to 30/70, which ratios take into account the solubility of the active substance chosen and of the film-forming agent chosen.

The nail varnishes according to the invention can additionally comprise additives commonly used in cosmetics, such as plasticizers based on phthalate, on camphor or on urea, colored pigments or dyes, pearlescent agents, antisettling agents, sulfonamide resins, silicates, fragrances, wetting agents, such as sodium dioctyl sulfosuccinate, lanolin derivatives, agents for protecting from light, such as 2-hydroxy-4-methoxybenzophenone, or substances possessing an antibacterial action.

The difficulty in the implementation of this invention has consisted in choosing film-forming agents which are insoluble in water but soluble in the water/alcohol mixture, so that, after drying, the thin film formed on the nail is insoluble in water and thus able to withstand the action of washing.

The nail varnish according to the invention is also suitable for prophylactic use against mycoses of the nail, it being possible for a sufficiently great deposited layer of active substance to be obtained on the nail so that, in the case of fungal contamination, the appearance of a disease of the nail brought about by fungi is not observed.

With the nail varnish according to the invention, it is possible to result in a complete recovery in the treatment of mycoses of the nails, the nails generally regrowing without deformation.

The present invention is illustrated in more detail with the help of the following examples.

### EXAMPLE 1

### Quantitative Compositions

| | | | | |
|---|---|---|---|---|
| Ciclopirox | 8 | g | 8 | g |
| Urea | 5 | g | 10 | g |
| Acrylic/methacrylic copolymer | 14 | g | 10 | g |
| Ethyl alcohol | 59.10 | g | 57.60 | g |
| Purified water, q.s. for | 100 | g | 100 | g |
| Ciclopirox | 6 | g | 6 | g |
| Urea | 5 | g | 10 | g |
| Acrylic/methacrylic copolymer | 14 | g | 10 | g |
| Ethyl alcohol | 59.10 | g | 59.20 | g |
| Purified water, q.s. for | 100 | g | 100 | g |

### EXAMPLE 2

### Measurement of the Antimycotic Activity

1. Procedure

The activity of the compounds used according to the invention was demonstrated by penetration tests on bovine horn. These bovine horns were cut up so as to obtain keratin disks with a diameter of approximately 1 cm and with a standardized thickness of 0.6 mm. These keratin disks were inoculated with a dermatophyte (Trichophyton mentagrophytes). After a period of approximately 30 days, these dermatophytes completely colonized the disks of horns, both at the surface and in the middle of the substance, perfectly mimicking the phenomenon of mycosis of the nail.

It is considered that a level of starting microorganisms of 7.46 log 10 spores/gram of horn is a level which is sufficiently high to make it possible to calculate the fall in log 10. The varnish comprising the ciclopirox is then deposited on this horn disk 5 days out of 7 and, on the seventh day, the horn is reduced to dust in order to be able to count the residual populations as colony-forming units.

This treatment was carried out for 1 month with sampling being carried out at T7 days, T14 days and T21 days.

The passage through the nail is studied by monitoring the disappearance of these dermatophytes.

This method makes it possible both to determine the passage through the nail of the active principle and its effectiveness in vitro.

A control of vigor of the strain is performed in parallel. The logarithmic reduction in the number of colony-forming units is demonstrated after neutralization of the varnish (a preliminary test made it possible to validate the neutralization of the formulation for this microorganism).

In the tests on the penetrating capability, several formulations, prepared either with film-forming agents possessing a hydrophilic tendency dissolved in aqueous/alcoholic media or with film-forming agents possessing a hydrophobic tendency dissolved in polar solvents, were tested.

The following formulations were tested:

Formulation No. 1

| | |
|---|---|
| Ciclopirox | 8 g |
| Acrylic/methacrylic copolymer | 14 g |
| Ethyl alcohol | 59.1 g |
| Purified water, q.s. for | 100 g |

Formulation No. 2

| | |
|---|---|
| Ciclopirox | 6 g |
| Urea | 5 g |
| Acrylic/methacrylic copolymer | 14 g |
| Ethyl alcohol | 59.1 g |
| Purified water, q.s. for | 100 g |

Formulation No. 3

| | |
|---|---|
| Ciclopirox | 8 g |
| Urea | 5 g |
| Acrylic/methacrylic copolymer | 14 g |
| Ethyl alcohol | 59.1 g |
| Purified water, q.s. for | 100 g |

2. Results

The results obtained on the 7th day, on the 14th day and on the 21st day are summarized in the following table.

Number of spores/g of horn (n = 6)
Time

| | T0 | T7 days | T14 days | T21 days |
|---|---|---|---|---|
| Control | 7.46 ± 0.3 | 7.9 ± 0.37 | 7.67 ± 0.2 | 7.56 ± 0.16 |
| F₁ | 7.46 ± 0.3 | 4.34 ± 3.02 | 4.18 ± 2.69 | 3.02 ± 2.20 |
| F₂ | 7.46 ± 0.3 | 1.06 ± 0.73 | 3.62 ± 0.88 | 0.59 ± 0.87 |
| F₃ | 7.46 ± 0.3 | 1.67 ± 1.42 | 1.31 ± 1.42 | 0.12 ± 0.29 |

It emerges from these studies that the formulations with urea (formulations 2 and 3) are the most effective as the kinetics for destruction of the dermatophytes are faster; a fall of 5.79 and 6.4 respectively for the formulations 3 and 2 is observed at 7 days, whereas the fall is only 3.12 for the urea-free formulation 1.

The fungicidal effect is achieved the most rapidly (in 7 days) for the formulations 2 and 3 and is complete at 21 days. On the other hand, the kinetics for destruction are much slower with the formulation 1, which does not comprise urea and for which the fungicidal effectiveness is not achieved after 21 days (fall of 4.44 in 21 days).

It is known that the surface horny layers have, inter alia, the biological role of preventing the penetration of foreign substances. The compositions according to the invention are distinguished from the preparations of the prior state of the art known for the treatment of the nails in that the surface horny layers allow a considerable proportion of the active substances present in these preparations to pass and in that these compositions according to the invention thus exert an effective, at depth, lasting action.

1-11. (canceled)

12. A nail varnish possessing an antimycotic action, comprising:
a) 2 to 80% by weight, with respect to the amount of volatile compounds, of at least one 1-hydroxy-2-pyridone of general formula (I) 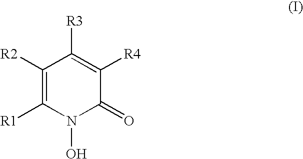
in which
R1 represents either a straight or branched alkyl group having from 6 to 9 carbon atoms or a cycloalkyl group having from 3 to 6 carbon atoms,
one of the substituents R2 and R4 represents a hydrogen atom and the other represents a hydrogen atom or a group chosen from the methyl and ethyl groups, and
R3 represents a straight or branched alkyl group having 1 or 2 carbon atoms,
in the free form or in the form of a physiologically acceptable salt, as substance possessing an antimycotic action,
b) from 0.1 to 20% by weight, with respect to the amount of volatile compounds, of a promoter of absorption of the active substance chosen from the group comprising urea, ammonium sulfite, esters and salts of thioglycolic acid, allantoin, enzymes and salicylic acid,
c) 5 to 40% by weight, with respect to the amount of volatile compounds, of a film-forming agent which is soluble in polar solvents, and optionally
d) a physiologically acceptable solvent, as well as, optionally,
e) additives acceptable in cosmetology.

13. The nail varnish as claimed in claim 12, wherein the active substance possessing an antimycotic action corresponds to the formula (I) in which R2 and R4 are each a hydrogen atom.

14. The nail varnish as claimed in claim 12, wherein the active substance possessing an antimycotic action corresponds to the formula (I) in which R1 represents a cycloalkyl group having from 3 to 6 carbon atoms.

15. The nail varnish as claimed in claim 14, wherein the active substance possessing an antimycotic action corresponds to the formula (I) in which R1 represents a cyclohexyl group.

16. The nail varnish as claimed in claim 12, wherein the active substance possessing an antimycotic action corresponds to the formula (I) in which R1 represents a —CH₂—CH(CH₃)—CH₂—C(CH₃)₃ group.

17. The nail varnish as claimed in claim 12, wherein the agent which promotes absorption of the active substance is urea.

18. The nail varnish as claimed in claim 12, wherein the active substance possessing an antimycotic action represents 10 to 60% by weight with respect to the amount of nonvolatile components.

19. The nail varnish as claimed in claim 18, wherein the active substance represents 20 to 40% by weight with respect to the amount of nonvolatile components.

20. The nail varnish as claimed in claim 12, wherein the active substance possessing an antimycotic action represents 0.5 to 20% by weight with respect to the amount of the nonvolatile components.

21. The nail varnish as claimed in claim 20, wherein the active substance possessing an antimycotic action represents 2 to 15% by weight with respect to the amount of the non volatile components.

22. The nail varnish as claimed in claim 12, wherein the film-forming agent is chosen from the group consisting of acrylic/methacrylic copolymers, polymethacrylate/butyl acrylates, acrylic/acrylate copolymers, polyvinyl alcohol, polyvinylpyrrolidones, polyvinylpyrrolidone/vinyl acetates and vinylpyrrolidone/dimethylaminoethyl methacrylates.

23. Then nail varnish as claimed in claim 12, wherein the solvent is composed of a mixture of the solvents having a boiling point of less than 100° C. and of solvents having a boiling point of between 100 and 150° C. and optionally of a solvent having a boiling point of between 150 and 200° C.

24. The nail varnish as claimed in claim 23, wherein the solvent is a hydrophilic system chosen from the group comprising the water/ethanol, water/isopropyl alcohol and water/butanol mixtures.

25. The nail varnish as claim in claim 243, wherein the proportion ranges from 10/90 to 30/70.
